(19) United States
(12) Patent Application Publication
(10) Pub. No.: US 2009/0312832 A1
(43) Pub. Date: Dec. 17, 2009

(54) SLIP LAYER DELIVERY CATHETER

(75) Inventor: DENNIS J. DELAP, Bloomington, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 12/482,975

(22) Filed: Jun. 11, 2009

Related U.S. Application Data
(60) Provisional application No. 61/061,255, filed on Jun. 13, 2008.

(51) Int. Cl. A61F 2/06; A61F 11/00

(52) U.S. Cl. 623/1.11; 606/108; 623/1.2

(57) ABSTRACT

A slip layer delivery catheter includes a slip layer delivery catheter includes an outer tube configured with a plurality of strips extending therefrom and terminating in a plurality of distal strip end ends, the plurality of strips defined by empty slotted regions disposed between adjacent strips. An inner tube is coaxially disposed in the outer tube. The inner tube includes a proximal inner tube portion and a distal inner tube portion connected to the distal strip ends. An endoluminal medical device is collapsibly disposed over the inner tube. The plurality of strips is folded back into the outer tube, concentrically orienting the device between the outer tube and the inner tube such that at least a portion of the strips are disposed between the outer tube and the ablumenal device side.

This application claims the benefit of priority under 35 U.S.C. §119(e) to U.S. Provisional Application No. 61/061,255, filed Jun. 13, 2008, which is hereby incorporated by reference in its entirety.

### TECHNICAL FIELD

This invention relates generally to a catheter for delivering medical devices in percutaneous interventional procedures, and more particularly, an endoluminal medical device delivery system and a method for making an endoluminal medical device delivery system for use in angioplasty procedures, stenting procedures, and other device placement procedures and their related devices.

### BACKGROUND

Percutaneous interventional angioplasty procedures typically involve guide catheters introduced into the cardiovascular system and advanced through the aorta into a desired coronary artery. Using fluoroscopy, a guide wire is then advanced through the guide catheter and across an artery site to be treated, such as a blockage, lesion, stenosis, or thrombus in an artery lumen. A delivery catheter may then be advanced over the guide wire to deliver a suitable endoluminal medical device, such as a stent, graft, stent-graft, vena cava filter, or other vascular implant. In many cases, a stent is delivered to the treatment site to reinforce body vessels, keep the vessel open and unoccluded, and prevent restenosis. The stent is expanded to a predetermined size, thereby dilating the vessel so as to, for example, radially compress an atherosclerotic plaque in a lesion against the inside of the artery wall. The stent may be a mechanically expandable stent that is expanded using a balloon catheter, for example, or it may be a radially self-expanding stent utilizing resilient or shape memory materials, such as spring steel or nitinol. With respect to a balloon expandable stent, the stent is compressed or crimped about a balloon on the distal end of the catheter. The stent may be covered by an overlying sheath or sleeve to prevent the stent from becoming dislodged from the balloon. With respect to a self-expanding stent, the stent is positioned at a distal catheter end around a core lumen where it is held down (compressed) and covered by an overlying delivery sheath or sleeve. In either case, upon retraction of the sleeve, the stent is able to self-expand or be expanded with a balloon.

During the loading and deployment of self-expanding stents, there may be significant frictional forces between the stent surface and the surrounding delivery sheath. These forces may damage the coatings on coated stents, especially longer coated stents, and can create difficulties for sheath retraction and placement. The frictional forces can cause the stent to act like a spring, releasing the stored frictional forces beyond the sheath end and causing the stent to move or “jump” from the desired position and be imprecisely deployed. In addition to the imprecise placement of self-expanding stents, it is often difficult to predict the final stent length in advance of its expansion in the vessel. Further, once a portion of the stent has expanded against the vessel walls, it becomes difficult to adjust its position. Similar problems may occur during the loading and deployment of balloon expandable stents. For example, frictional forces between the protective sheath and the stent may damage any coating on the stent.

Accordingly, there is a need for a reliable endoluminal medical device delivery system, which addresses the above difficulties

### SUMMARY

In one aspect, a slip layer delivery catheter includes an outer tube configured with a plurality of strips extending from a distal end thereof and terminating in a plurality of distal strip end portions. The plurality of strips is defined by a plurality of empty slotted regions between adjacent strips. Each strip comprises a proximal and distal strip portion. An inner tube having a proximal inner tube portion and a distal inner tube portion is coaxially disposed in the outer tube. The distal tube portion is connected to the distal strip portions and an endoluminal medical device is collapsibly disposed over the inner tube. The strips are folded back into the outer tube, concentrically orienting the device between the outer tube and the inner tube such that at least a portion of the strips are disposed between the outer tube and the ablumenal device side. An atraumatic tip may be coupled to the distal end of the delivery catheter or to the distal end of the inner tube. The outer tube may further include a reinforcing coil proximal to the device.

In one embodiment, the distal strips are continuous with at least one layer of the outer tube, the plurality of strips directly extending from the at least one layer. The distal strip ends may be bonded to the inner tube distal to a collapsibly disposed medical device or at a position under the collapsibly disposed medical device. Alternatively, the distal strip ends may be bonded to the inner tube proximal to the collapsibly disposed medical device.

The distal strip portions may be disposed between at least a portion of inner tube and the lumenal device side. The proximal strip portions may extend between the outer tube and the ablumenal device side over the entire longitudinal length of the device. Similarly, the distal strip portions may extend between the inner tube and the lumenal device side over the entire longitudinal length of the device. Each of the plurality of strips may have a length between about one to about five times the longitudinal length of the medical device when compressively disposed within the outer tube. In addition, the plurality of strips may have a combined width less than the inner circumference of the medical device when compressively disposed within the outer tube.

Generally, each of the strips will be made from or include a low friction material, and preferentially a low friction material having a coefficient of friction less than about 0.1. In one embodiment, the low friction material forms an extruded polymeric outer layer. In another embodiment, the low friction material is formed as a coating applied to surfaces of the strips by spray coating or dip coating. In a particular embodiment, the low friction material is a polytetrafluoroethylene polymer. In a another embodiment, the low friction material may be an ultra-high molecular weight polyethylene polymer having a molecular weight between about 1 to about 10 million.

Suitable devices for use in the medical device delivery system of the present invention include mechanically expandable and self-expanding medical devices, including covered and uncovered stents, drug-eluting stents, stent grafts, filters, including vena cava filters, valves, occlusion devices, and the like.

In a particular embodiment, a self-expanding stent delivery system includes an outer tube comprising at least one layer and configured with a plurality of strips comprising a low friction material and extending from a distal end of the outer tube, the strips extending continuously from the at least one layer. Each of the plurality of strips has a proximal strip portion and a distal strip portion terminating in a distal strip end, the plurality of strips being defined by plurality of empty slotted regions disposed between adjacent strips. An inner tube includes a proximal inner tube portion and a distal inner tube portion, the distal inner tube portion connected to the distal strip ends. A self-expanding stent is collapsibly disposed over the inner tube, the stent being concentrically oriented between the inner tube and the outer tube, whereby the strips are folded back into the outer tube such that the proximal strip portions are disposed between the outer tube and the outer stent side and the distal strip portions are disposed between at least a portion of the inner tube and the inner stent side.

In another aspect, a method for fabricating a medical device delivery system includes providing an outer tube; forming a plurality of strips in a distal end of the outer tube by removing portions of the outer tube to form a plurality of empty slotted regions between adjacent strips. An inner tube is co-axially disposed within the outer tube. Each of the plurality of distal strip ends is connected to a distal portion of the inner tube. Prior to connecting the strip ends to the inner tube, the proximal strip portions may be preformed using shape memory materials to preferentially adopt a strip conformation whereby the strips are biased toward curling back so as to promote separation of the strips from the device during deployment. A portion of the inner tube is overlayed with an expandable medical device. At least a portion of each of the inner tube, the device, and the plurality of strips is translocated into the outer tube, such that at least a portion of the strips are disposed between the outer tube and an external side of the device.

In a further aspect, a method for using the above described system to deploy a device, such as a stent, includes extending a guidewire through a vascular or bodily lumen to a desired device placement site. The delivery catheter is then advanced over and along the guidewire such that the corresponding position of the device is spaced within the desired device placement site. At this point, the outer tube may be retracted in a proximal direction relative to the inner tube, which causes the proximal strip portions to unfurl from the distal end of the outer tube and peel away from the ablumenal side of the device to expose and allow from mechanical expansion or self-expansion of the device within the vascular or bodily lumen at the desired site. Upon placement and expansion of the device, the delivery catheter is removed from the vascular lumen or bodily opening.

Advantageously, the present invention is believed to: minimize difficulties associated with retraction of the outer tube or advancement of the inner tube during deployment of the device; reduce frictional forces between surfaces on the device and surfaces on the inner and outer tubes, thereby enhancing more accurate placement of the device; better preserve the integrity of surface coatings on devices, such as coatings on drug-eluting stents; allow for accurate placement and delivery of larger devices, including longer stents between about 120-240 mm in length or longer; and simplify fabrication of the above delivery system.

### BRIEF DESCRIPTION OF THE DRAWINGS

The accompanying drawings incorporated in and forming a part of the specification illustrate several aspects of the present invention, and together with the description serve to explain the principles of the invention. In the drawings:

FIG. 1 is a side view of an endoluminal medical device delivery system of the type described in the present invention.

FIG. 2A is a partial side sectional view of an endoluminal medical device delivery system according to one embodiment of the present invention.

FIG. 2B is a cross-sectional view of the endoluminal medical device delivery system depicted in FIG. 2A and taken along line 2B-2B;

FIG. 2C is a cross-sectional view of the endoluminal medical device delivery system depicted in FIG. 2A and taken along line 2C-2C.

FIG. 3A is a partial side sectional view of an endoluminal medical device delivery system according to another embodiment of the present invention;

FIG. 3B is a cross-sectional view of the endoluminal medical device delivery system depicted in FIG. 3A and taken along line 3B-3B.

FIG. 4 is a side sectional view in which the outer tube of the endoluminal medical device delivery system in FIG. 2A is retracted so as to release the compressibly disposed stent.

### DETAILED DESCRIPTION

The term “endoluminal medical device” refers to covered and uncovered stents, filters, and any other device that may be implanted in a vascular or bodily lumen or opening in a patient including, for example, a human artery.

The terms “proximal” and “distal” refer to a direction closer to or away from, respectively, an operator (e.g., surgeon, physician, nurse, technician, etc.) who would insert the medical device into a patient, with the tip-end (i.e., distal end) of the device inserted inside a patient's body. Thus, for example, a “proximal portion” would refer to a medical device portion closer to the operator, while a “distal portion” would refer to a medical device portion further away from the operator toward the tip-end of the device.

The term “stent” refers to a device or structure that provides or is configured to provide rigidity, expansion force, or support to a body part, for example, a diseased or otherwise compromised body lumen.

The term “self-expandable” refers to a resilient object, device, or structure having a radially constrained lower diameter configuration when compressed inside a tube or sheath that is capable expanding to form a desired radially-expanded diameter when unconstrained, i.e. released from the radially constraining forces of a tube or sheath, without application of an externally added force.

The term “mechanically expandable” refers to a device that comprises a reduced profile configuration and an expanded profile configuration, and may undergo a transition from the reduced configuration to the expanded configuration via an outward radial mechanical force, such as, for example, from a balloon expanded by a suitable inflation medium, or any other mechanism including but not limited to those employing mechanical, hydraulic, and/or pneumatic techniques.

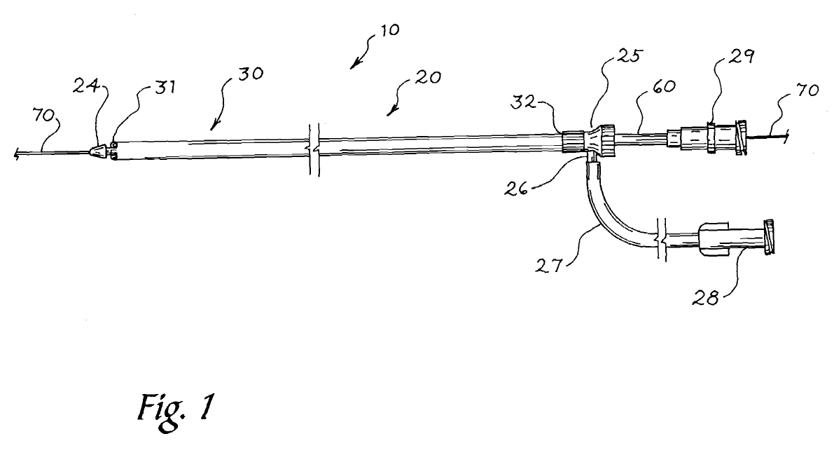

Turning now to the drawings, FIG. 1 depicts an exemplary endoluminal medical device delivery system 10, including a delivery catheter 20. The delivery catheter 20 includes an outer tube 30 with a distal end 31 and a proximal end 32. The delivery catheter 20 further includes an inner tube 60 extending longitudinally though an inner passageway of the outer tube 30. The inner tube 60 is connected to a tapered distal tip 24 for accessing and dilating a vascular access site over a guidewire 70, which extends through a lumen of the inner tube 60. The general configuration of the delivery catheter 20 in FIG. 1 is typical of introducer catheters or sheaths known in the art.

In FIG. 1, a connector valve 25, attached about the proximal end 32 of the outer tube 30, typically includes one or more silicone disks (not shown) for preventing the backflow of fluids therethrough. The disks typically include a slit or aperture to allow for passage of the inner tube 60 therethrough. The connector valve 25 also includes a side arm 26 to which a tube 27 and male Luer lock connector 28 may be connected for introducing and/or aspirating fluids through the delivery catheter 20. A guidewire 70 can be inserted in the vessel with an introducer needle using, for example, the well-known percutaneous vascular access Seldinger technique. A male Luer lock connector hub 29 is attached at the proximal inner tube end 62 for connection to syringes and other medical apparatuses.

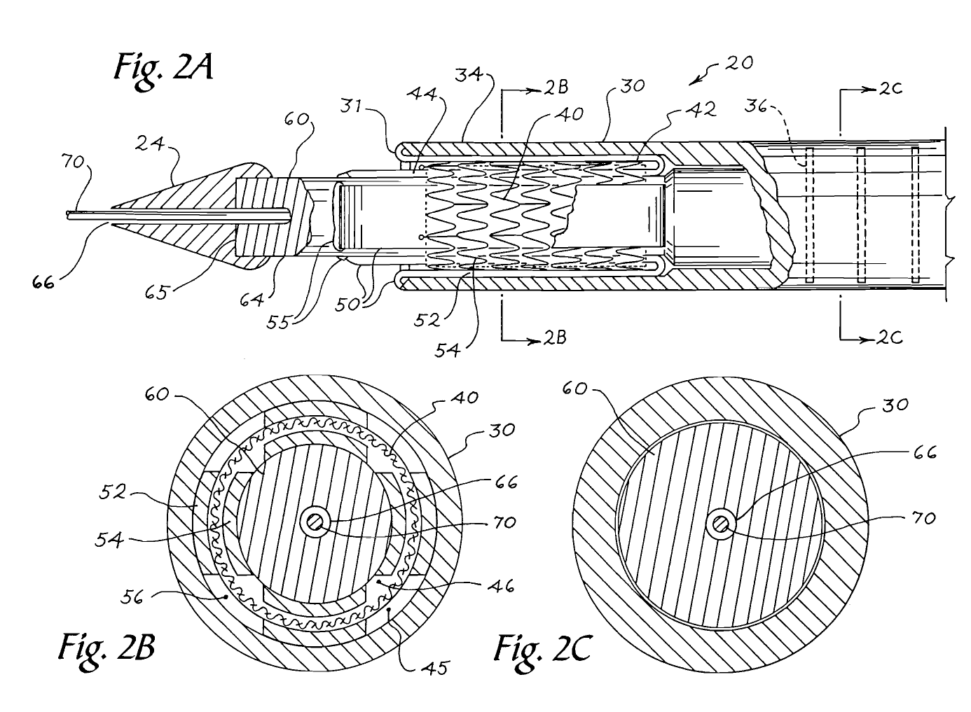

FIGS. 2A and 2B depict the distal end of the slip layer delivery catheter 20 housing an endoluminal medical device 40, which is depicted in FIG. 2A as a self-expanding stent. The device 40 is defined by a proximal device end 42 and distal device end 44. The slip layer delivery catheter 20 includes an outer tube 30, including a distal outer tube portion 34 and a distal outer tube end 31. The distal outer tube end 31 is coupled to a plurality of strips 50 extending from the distal strip end 31, including proximal strip portions 52 extending over the self-expanding stent 40 and distal strip portions 54 extending under the stent 40, each strip 50 terminating at a distal strip end 55. The delivery catheter 20 further includes a coaxially disposed inner tube 60 having a distal inner tube portion 64, including an inner tube distal end 65. The distal inner tube end 65 is coupled to a tapered tip 24.

As shown in FIGS. 2A and 2B, the self-expanding stent 40 is concentrically mounted over the inner tube 60 and the distal strip portions 54. When the device 40 is compressibly disposed in the delivery catheter 20 in a retracted state (as shown), the distal inner tube portion 64 or inner tube distal end 65 is disposed or adjacent to the distal end 31 of the outer tube 30 such that the strips 50 are folded within the outer tube 30. In this configuration, the stent 40 is concentrically oriented or sandwiched between the outer tube 30 and the inner tube 60 such that the proximal strip portions 52 are disposed between the outer tube 30 and the ablumenal (outer) device side 45 and the distal strip portions 54 are disposed between the inner tube 60 and the lumenal (inner) device side 46.

In one embodiment, the device 40 and an inner tube distal portion 64 or inner tube distal end 65 are positioned relative to the outer tube 30 so that the proximal strip portions 52 between the outer tube 30 and the ablumenal device side 45 extend over only a part of the longitudinal length of the device or stent 40. In another embodiment, the device 40 and the inner tube distal portion 64 or inner tube distal end 65 are positioned relative to the outer tube 30 SO that the proximal strip portions 52 between the outer tube 30 and the ablumenal device side 45 extend over the entire longitudinal length of the device or stent 40.

The distal strip ends 55 may be attached to the inner tube 60 proximal or distal to the distal device end 44. In particular, the distal strip ends 55 may be attached to the inner tube 60 directly under the collapsibly disposed device 40, at or near the distal device end 44, or distal to the distal device end 44. Accordingly, the distal strip portions 54 may be disposed between the luminal side device side 46 and the inner tube 60 over some or all of the device's longitudinal length.

FIGS. 2A and 2B depict an exemplary embodiment in which the proximal strip portions 52 disposed between the outer tube 30 and the ablumenal device side 45 extend over the entire longitudinal length of the stent 40 and the distal strip portions 54 disposed between the inner tube 60 and lumenal device side 46 extend over the entire longitudinal length of the stent 40. In accordance with this embodiment, the strips 50 are connected to the inner tube 60 at positions distal to the device 40 and proximal to inner tube distal end 65 and then folded back into the outer tube 30 so as to completely envelope (or form a covering) over the ablumenal 45 and lumenal 46 sides of the device 40.

Alternatively, the strips 50 may be connected to the inner tube 60 proximal to the device 40 and then folded back into the outer tube 30 to only cover the ablumenal 45 side of the device 40. Thus, the strips may envelope in part, or in whole, one or both sides of the device 40.

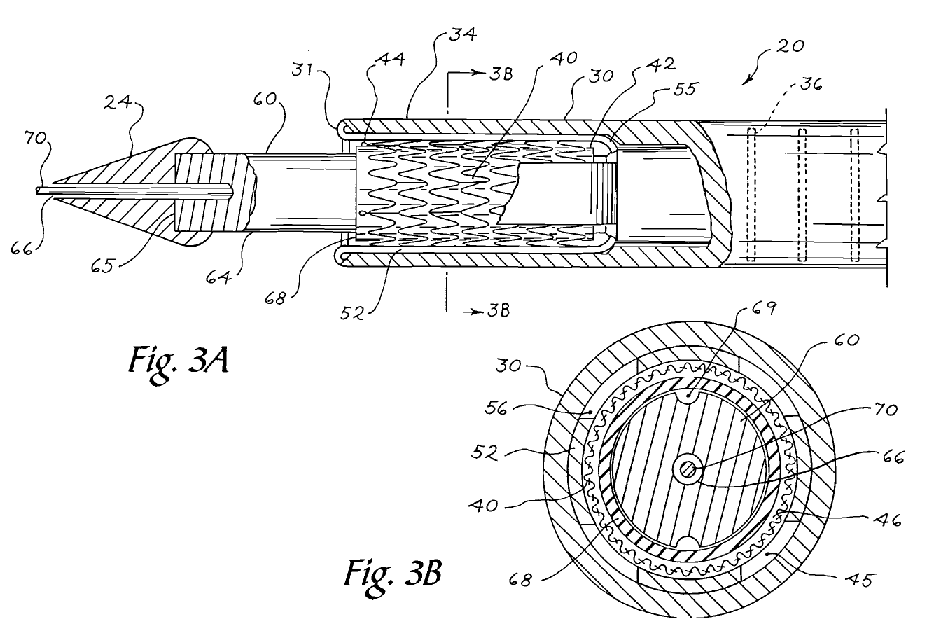

For example, in the embodiment shown in FIG. 3A and 3B, the strips are configured to envelope only the outside ablumenal side 45 of the device 40, which is illustrated as a balloon expandable stent 40. Since the delivery system 10 of this embodiment is configured to deploy a balloon expandable stent 40, the inner tube 60 will include an expandable member, such as a balloon 68, connected to one or more inflation lumens 69 (FIG. 3B) for expanding the stent 40. Thus, it may be preferable to only cover the ablumenal 45 side of a device 40, whereby the distal strip ends 55 are bonded to the inner tube 60 at positions proximal to the balloon expandable stent 40, so as to not interfere with the expansion of the balloon 68. This configuration may also reduce the overall diameter of the delivery system 10.

The inner tube 60 may be further defined by a hollow channel 66 accommodating entry of a guidewire 70 therethrough for purposes of advancing the delivery catheter 20 to a predetermined position in a bodily lumen or vessel to facilitate delivery of the device 40 by conventional percutaneous delivery means.

FIG. 2A depicts a tapered tip 24 coupled to the inner tube distal end 65. The tip 24 is generally formed from a soft material, such as a soft polymer capable of being bonded to the inner tube 60. Preferably, the tip 24 is tapered and/or rounded to facilitate an atraumatic entry into and through a bodily lumen. The tip 24 may further include barium sulfate, gold, or other suitable radioapaque and/or MRI contrast agents known to those of skill in the art for fluoroscopic device imaging.

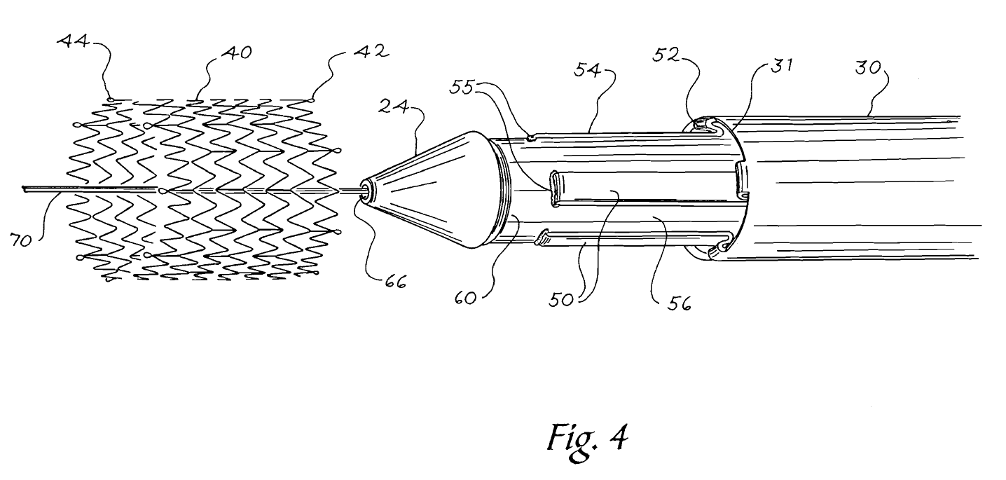

The strips 50 are made from or include a surface material having a low coefficient of friction, so that when positioned between the device 40 and the outer 30 and/or inner 60 tubes, the strips 50 are capable of reducing the frictional forces engaging these elements during deployment of the device 40 so as to enhance, for example, retraction of the outer tube 30 and/or advancement of the inner tube 60 to facilitate deployment of a lumenally positioned device 40 against a desired vessel wall region (FIG. 4). More particularly, it is believed that configuring the strips 50 as described above: (1) minimizes the difficulties associated with retraction of the outer tube 30 or advancement of the inner tube 60 during deployment of the device 40; (2) reduces the frictional forces between surfaces on the device 40 and surfaces on the inner and outer tubes 60, 30, respectively, thereby enhancing more accurate placement of the device; (3) helps to better preserve the integrity of surface coatings on devices, such as coatings on drug-eluting stents; and (4) allows for accurate placement and delivery of larger devices, including longer stents between about 120-240 mm in length or longer.

In one embodiment, the strips 50 extend continuously from the outer tube distal end 46. Thus, and as best seen in FIG. 4, the strips 50 can constitute extensions from the outer tube 30 that can be formed by cutting a plurality of slotted regions 56 from the distal outer tube portion 34 and removing the portions of the outer tube 30 from the slotted regions 56, thereby forming a series of strips 50 extending from the outer tube 30. Where a multilayer outer tube 30 is used, the strips 50 may be continuous with one or more layers. Thus, the strips 50 may be continuous with the outer (ablumenal) layer only or they may be continuous with the outer tube 60 wall or sheath as a whole. Alternatively, the strips 50 may be separately coupled or attached to the distal outer tube portion 34 or outer tube end 31 at a proximal plurality of strip ends and to a distal inner tube portion 64 or distal inner tube end 65 at a distal plurality of strip ends.

In one aspect, the strips 50 may be formed to have a length between about one to about five times the longitudinal length of the device 40 when compressively disposed in the outer tube 30. In another aspect, the plurality of strips 50 may be configured to have a combined width less than the inner circumference of the medical device when compressively disposed in the outer tube. In a particular embodiment, the strips 50 are configured to maximize the degree of contact between the device 40 and the strips 50 without negatively impacting the ability to retract or contract the outer and inner tubes 30, 60 during system assembly or during device deployment.

In a further aspect, the strips 50 may be preformed using shape memory materials to promote separation of the strips from the device 40 during deployment. The shape memory material may include a polymer material capable of retaining a predetermined configuration or shape using conventional heat-treatment techniques. Preferably, the strips 50 are preformed prior to their attachment to the inner tube 60. In particular embodiment, the proximal strip portions 52 are preformed to preferentially adopt a strip conformation during deployment, whereby the proximal strip portions 52 are biased toward curling back away from the device 40, promoting separation of the strips 50 from the device 40 such that deployment and/or self-expansion of the device 40 is unimpeded by the proximal strip portions 52. In this case, the use of preformed, shape memory strip portions 52 may reduce, for example, pinching of the strips 50 upon deployment and/or expansion of the device 40.

The outer and inner tubes 30, 60 are tubular elongate structures that can each be fabricated from multiple materials by conventional co-extrusion processes to form a single layer tube or as a multi-layer tube. Additional layers may be included to provide a desired level of flexibility or stiffness. Accordingly, the outer and inner tubes 30, 60 may be constructed by processes employing single-layer or multiple-layer extrusion; braid coil, stacked coil, or coil-reinforced extrusion; and combinations thereof incorporating a variety of polymeric and/or other suitable materials. In addition, portions of the outer and inner tubes 30, 60 may be tapered in or tapered out as depicted in FIG. 2. The outer and inner tubes 30, 60, and the strips 50 may be formed from polymers or polymeric composites. Alternatively, they may be formed from or include non-polymeric materials as well.

In one embodiment, the low friction materials are extruded as a surface layer in a single- or multilayer outer tube 30 from which the plurality of strips 50 are derived. Exemplary low friction materials include fluoropolymers, including polytetrafluoroethylene (PTFE), tetrafluorethyleneperfluorpropylene (FEP), perfluorollkoxy (PFA) copolymer, ethylenetetrafluoroethylene (ETFE), PTFE/PFA blends, amorphous fluoropolymers (AFs), and various DuPont Teflon® resins, combinations, blends, and coatings thereof; high density polyethylene (HDPE), melt-extrudable ultra-high molecular weight polyethylenes (UHMWPEs) having a molecular weight between about 1 to about 10 million, including linear polyolefin resins GUR®5113 and Hostalloy®731 (Ticona), polypropylene polyolefin materials, nylon materials, polyurethane elastomers, including Pellethane™, including combinations and blends thereof. The low friction materials may be coated or extruded as low friction surface layers covering the entire length of a tube or only covering specific tube portions. A low friction surface layer may include homopolymers, copolymers, polymeric blends or combinations thereof.

The low friction strip materials may include or be configured as preformed shape memory materials, as described above. The preformed shape memory materials may be configured from PTFE, as well as a variety other shape memory polymeric material known to those of skill in the art.

In another embodiment, the low friction materials may be applied to one or more surface(s) of the tubes 30, 60 or strips 50 as a lubricious surface coating by spray coating, dip coating, powder coatings, and other methods known to those of skill in the art. Lubricious surface coatings may include the above described low friction polymers, including hydrophobic or hydrophilic fluoropolymer-based coatings (including PTFE), a variety of hydrophilic coatings, including silicone-based coatings, water-based polyurethane coatings, heparinized coatings, polyvinylpirilidone (PVP)-based coatings, hydrogels, BIOSLIDE™ (SciMed Life Systems, Inc., Maple Grove Minn.), MICROGLIDE™ (Advanced Cardiovascular Systems), amorphous diamond coatings, and the like.

Hydrophilic coatings may include a hydrophilic polymer selected from the group comprising polyacrylate, copolymers comprising acrylic acid, polymethacrylate, polyacrylamide, poly(vinyl alcohol), poly(ethylene oxide), poly(ethylene imine), carboxymethylcellulose, methylcellulose, poly(acrylamide sulphonic acid), polyacrylonitrile, poly(vinyl pyrrolidone), agar, dextran, dextrin, carrageenan, xanthan, and guar. The hydrophilic polymers can comprise ionizable groups such as acid groups, e.g., carboxylic, sulphonic or nitric groups. The hydrophilic polymers may be cross-linked through a suitable cross-binding compound. A cross-binder generally comprises two or more functional groups which provide for the connection of the hydrophilic polymer chains. The choice of cross-binder can depend on the polymer system: if the polymer system is polymerized as a free radical polymerization, a preferred cross-binder comprises 2 or 3 unsaturated double bonds.

Low friction surface materials for use in the present invention may be chosen to exhibit a coefficient of friction less than 0.25, preferably less than about 0.15, more preferably less than about 0.1, and most preferably less than about 0.05. An Imass Slip/Peel Tester Model SP-2100 (Imass, Inc., Accord, Mass.) may be used to quantitatively determine coefficient of friction data for a given surface material or coating using the ASTM method D-1894.

The low friction materials described above may be coated onto or extruded into any of the above described delivery system 10 components, including any of the surfaces in the outer tube 30, strips 50, inner tube 60, device 40, guidewire 70, and/or tip 24.

Additional polymeric materials or resins used to make the outer and inner tubes 30, 60 include hydrophilic polyurethanes, aromatic polyurethanes, polycarbonate base aliphatic polyurethanes, engineering polyurethane, elastomeric polyamides, block polyamide/ethers, polyether block amide (PEBA), including PEBAX®, silicones, polyether-esters, polyether-ester elastomers, including Arnitel® (DSM Engineering Plastics), nylons, polyesters, polyester elastomers, including Hytrel®) (Du Pont), linear low density polyethylenes, such as Rexell®, and combinations thereof.

Any one of the outer and inner tubes 30, 60 or strips 50 may further include a matrix of materials conventionally used in catheters, including reinforcing coils or other supportive materials within, external to or internal to such a matrix. The matrix of materials of materials and/or multilayer construct may be prepared in a variety of catheter configurations for producing a desired level of flexibility or stiffness for a given length of tube.

FIGS. 2A and 2B depict a tapered outer tube 30 having a multilayer configuration, including an outer layer 35 comprising a low friction material, such as PTFE; a middle layer comprising a stainless steel circumferential spiral reinforcing coil 36; and inner layer comprising a low friction material or another tube material, such as polyether block amide (PEBA) or nylon. In this configuration, the coil 36 may provide a desired stiffness to proximal tube portions and more flexibility to distal tube portions in which the device 40 is constrained. Alternatively, the reinforcing coil 36 may alternatively or additionally extend over the area housing the stent 40. This can provide the additional radial strength to constrain the stent 40 over long periods of storage time and reduce the chances of the stent 40 becoming increasingly embedded in the inner surface of the outer tube 30 so as to potentially interfere with retraction of the outer tube 30 during deployment.

Whether alone or blended with other materials, other reinforcing materials may include polyamides, including Durethan® (Bayer) and Cristamid® (ELF Atochem), polyethylene (PE), polypropylenes (PP), high-density polyethylene (HDPE), polyetheretherketone (PEEK), polyimide (PI), polyetherimide (PEI), liquid crystal polymers (LCP), and acetal polymers, including Delrin® and Celcon®.

The delivery system 10 of the present invention may be used to accommodate a variety of radially expandable, including self-expandable luminal devices. Exemplary endoluminal devices include stents, stent grafts, filters, including vena cava filters, valves, occlusion devices, and the like.

Stents, including self-expanding stents can be made of stainless steel, materials with elastic memory properties, such as NITINOL, or any other suitable material. Exemplary self-expanding stents include Z-STENTS™ and ZILVER™ stents, which are available from Cook Incorporated, Bloomington, Ind. USA. Balloon-expandable stents may be made, for example, of stainless steel (typically 316LSS, CoCr, etc.). Hybrid stents may be provided by combining one or more self-expanding stents or stent portions with one or more balloon-expandable stents or stent portions.

A stent may be bare, or it may include a drug coating, such as a coated drug-eluting stent or it may include a covering or graft material, such as stent graft.

Coated drug-eluting stents in the present invention may include a variety of materials for facilitating controlled drug release, including porous polymeric coating layers (US 2007/0150047 A1, 2003/0028243 A1, 2003/0036794 A1, and U.S. Pat. No. 6,774,278 B1), biodegradable elastomeric coating layers (US 2007/0196423 A1), drug coatings (US 2008/0020013 A1, 2007/0212394 A1), porous structures (US 2007/0073385 A1), or surface roughened or textured surfaces (U.S. Pat. No. 6,918,927 B2), the patent disclosures of which are incorporated by reference herein.

Suitable coverings or graft materials for stent grafts may include natural biomaterials, biocompatible polymers, and combinations thereof. Exemplary biocompatible polymers for use in stent grafts include poly(ethylene terephthalate), polylactide, polyglycolide and copolymers thereof; fluorinated polymers, such as polytetrafluoroethylene (PTFE), expanded PTFE and poly(vinylidene fluoride); polysiloxanes, including polydimethyl siloxane; and polyurethanes, including polyetherurethanes, polyurethane ureas, polyetherurethane ureas, polyurethanes containing carbonate linkages and polyurethanes containing siloxane segments, and combinations thereof, the disclosures of which are disclosed in U.S. Pat. Appl. Nos. 2006/0009835 A1, 2005/0159804 A1, and 2005/0159803 A1, the disclosures of which are incorporated by reference herein. Exemplary biomaterials for use in stent grafts of the present invention include collagen and extracellular matrix materials as described in U.S. Pat. No. 7,244,444 B2, the disclosure of which is incorporated by reference herein.

In a further aspect, a method for fabricating a medical device delivery system 10 includes providing an outer tube 30; forming a plurality of strips 50 in a distal end 31 of the outer tube 30 by removing portions of the outer tube to form a plurality of empty slotted regions 56 between adjacent strips 50. An inner tube 60 is co-axially disposed within the outer tube 30. Each of the plurality of distal strip ends 55 is connected to a distal portion 64 of the inner tube 60. Prior to connecting the strip ends 55 to the inner tube 60, the proximal strip portions 52 may be preformed using shape memory materials to preferentially adopt a strip conformation whereby the strips 50 are biased toward curling back so as to promote separation of the strips 50 from the device 40 during deployment. A portion of the inner tube 60 is overlayed with an expandable medical device. At least a portion of each of the inner tube, the device, and the plurality of strips is translocated into the outer tube, such that at least a portion of the strips are disposed between the outer tube and an external side of the device

The distal strip ends 55 may be connected to the inner tube 60 at a position distal to, under, or proximal to the device 40. A method for fabricating a mechanically expandable device 40, such as balloon expandable stent, may further include, for example, attaching a balloon 68 around the inner tube 60 below the device 40; and incorporating one or more inflation lumens 69 through the inner tube, which are connectively linked to the balloon 68, whereby the distal strip ends 55 are connected proximal to the device 40 as described above, and as shown in FIGS. 3A and 3B.

In a further aspect, the present invention includes a method for using the above described system 10 to deploy a device 40. More particularly, when deploying a device 40, such as a stent, the guidewire 70 is extended through a vascular or bodily lumen to a desired device placement site. The delivery catheter 20 is then advanced over and along the guidewire 70 such that the corresponding position of the device 40 is spaced within the desired device placement site. At this point, the outer tube 30 may be retracted in a proximal direction relative to the inner tube 60, which causes the proximal strip portions 52 to unfurl from the distal end of the outer tube and peel away from the ablumenal side 45 of the device 40 to expose and allow from mechanical expansion or self-expansion of the device 40 within the vascular or bodily lumen at the desired site. Upon placement and expansion of the device 40, the delivery catheter 20 is removed from the vascular lumen or bodily opening.

Of course, it will be recognized by those skilled in the art that many different sizes and types of catheters and catheter materials may be employed in conjunction with the present invention, including any of those disclosed in, all of which are expressly incorporated by reference herein.

The foregoing description of various embodiments of the invention has been presented for purposes of illustration and description. It is not intended to be exhaustive or to limit the invention to the precise embodiments disclosed. Numerous modifications or variations are possible in light of the above teachings. The embodiments discussed were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

1. A self-expanding stent delivery system comprising:
an outer tube comprising at least one layer and configured with a plurality of strips comprising a low friction material and extending from a distal end of the outer tube, the strips extending continuously from the at least one layer, each of the plurality of strips comprising a proximal and distal strip portion and terminating in a distal strip end, the plurality of strips being defined by plurality of empty slotted regions disposed between adjacent strips;
an inner tube having a proximal inner tube portion and a distal inner tube portion, the distal inner tube portion connected to the distal strip ends; and
a self-expanding stent collapsibly disposed over the inner tube and concentrically oriented between the inner tube and the outer tube, the stent defined by an inner lumenal stent side and an outer ablumenal stent side,
wherein the strips are folded back into the outer tube such that the proximal strip portions are disposed between the outer tube and the ablumenal stent side and the distal strip portions are disposed between at least a portion of the inner tube and the lumenal stent side.

2. An endoluminal medical device delivery system comprising:
an outer tube configured with a plurality of strips extending therefrom and terminating in a plurality of distal strip ends, the plurality of strips defined by a plurality of empty slotted regions disposed between adjacent strips;
an inner tube having a proximal inner tube portion and a distal inner tube portion, the distal inner tube portion connected to the plurality of distal strip ends; and
a deployable endoluminal medical device collapsibly disposed over the inner tube, the device having an inner lumenal device side and an outer ablumenal device side,
wherein the strips are folded back into the outer tube, concentrically orienting the device between the outer tube and the inner tube such that at least a portion of the strips are disposed between the outer tube and the ablumenal device side.

3. The delivery system of claim 2, wherein the distal strips are continuous with at least one layer of the outer tube, the plurality of strips directly extending from the at least one layer.

4. The delivery system of claim 2, wherein the distal strip ends are bonded to the inner tube distal to the device.

5. The delivery system of claim 2, wherein the distal strip ends are bonded to the inner tube proximal to the medical device.

6. The delivery system of claim 2, wherein each of the plurality of strips comprises a proximal strip portion and a distal strip portion, wherein the proximal strip portion is disposed between the outer tube and the ablumenal device side and the distal strip portion is disposed between at least a portion of the inner tube and the lumenal device side.

7. The delivery system of claim 6, wherein the proximal strip portions extend between the outer tube and the ablumenal device side over the entire longitudinal length of the device and the distal strip portions extend between the inner tube and the lumenal device side over the entire longitudinal length of the device.

8. The delivery system of claim 2, wherein each of the plurality of strips has a length between about one to about five times the longitudinal length of the medical device.

9. The delivery system of claim 2, wherein the plurality of strips have a combined width less than the inner circumference of the medical device when collapsibly disposed within the outer tube.

10. The delivery system of claim 2, wherein the strips comprise a low friction material.

11. The delivery system of claim 10, wherein the low friction material forms an extruded polymeric outer layer.

12. The delivery system of claim 10, wherein the low friction material is formed as a coating applied to surfaces of the strips by spray coating or dip coating.

13. The delivery system of claim 2, wherein the low friction material comprises a polytetrafluoroethylene polymer.

14. The delivery system of claim 2, wherein the low friction material comprises an ultra-high molecular weight polyethylene polymer having a molecular weight between about 1 to about 10 million.

15. The delivery system of claim 2, wherein the low friction material comprises a coefficient of friction of less than about 0.1.

16. The delivery system of claim 2, wherein the outer tube comprises a multilayer catheter body comprising a reinforcing coil proximal to the device.

17. The delivery system of claim 2, further comprising an atraumatic tip coupled to the distal end of the delivery system.

18. The delivery system of claim 2, wherein the medical device is a self-expanding medical device.

19. The delivery system of claim 2, wherein the medical device is a stent.

20. A method for fabricating a medical device delivery system comprising:
providing an outer tube;
forming a plurality of strips in a distal end of the outer tube by removing portions of the outer tube to form a plurality of empty slotted regions between adjacent strips;
co-axially disposing an inner tube within the outer tube;
connecting a plurality of distal strip ends of the strips to a distal portion of the inner tube;
overlaying an expandable medical device over a portion of the inner tube; and
translocating a distal portion of the inner tube, the device, and the plurality of strips into the outer tube, such that at least a portion of the strips are disposed between the outer tube and an external side of the device.
